(19) United States
(12) Patent Application Publication
(10) Pub. No.: US 2012/0164087 A1
(43) Pub. Date: Jun. 28, 2012

(54) Human Serum Albumin-Based Topical Ointment for Treatment of Acne, Psoriasis, Egfr-Induced Toxicity, Premature Skin Aging and Other Skin Conditions

(76) Inventor: Daniel C. Carter, Fayetteville, TN (US)

(21) Appl. No.: 13/266,051

(22) PCT Filed: Apr. 26, 2010
(86) PCT No.: PCT/US10/32400

§ 371 Date: Feb. 17, 2012

Related U.S. Application Data
(60) Provisional application No. 61/172,461, filed on Apr. 24, 2009.

(51) Int. Cl. A61K 38/38; A61P 17/06; A61P 27/02; A61P 17/10; A61P 31/00; A61Q 17/04; A61K 38/47; A61P 17/00

(52) U.S. Cl. 424/60; 514/15.2; 424/94.61; 424/702; 424/59

(57) ABSTRACT

Dermatological compositions and method for treating psoriasis, eczema, acne and like skin conditions for sanitization pharmaceutical compounding and protection of the skin from extreme environmental conditions are provided which contain serum albumin in an amount effective to treat, reduce the symptoms and improve the appearance of affected skin due to psoriasis, eczema, and acne and like conditions, enhance the delivery performance or stability of pharmaceutical compounding bases, protect the skin from the environment and premature aging, and to lubricate and/or promote the healing of eye after surgical or accidental trauma, when combined with a suitable topical ointment, antibacterial or dermatological agent, pharmaceutical compounding ointment or bases, vehicle, carrier or excipient. The albumin compositions may be in any suitable form for treating skin, such as a cream, oil, lotion, gel, gel-based ointment, and the like. The serum albumin compositions are preferably prepared using recombinant human serum albumin, a truncated version or fragment thereof.

### CROSS REFERENCE TO RELATED APPLICATIONS

This application claims the benefit of U.S. Provisional Application Ser. No. 61/172,461, filed Apr. 24, 2009, incorporated herein by reference.

### FIELD OF THE INVENTION

This invention relates in general to the use of a serum albumin composition to promote health, protective maintenance and treatment of a spectrum of skin conditions including those involving the eye, and more specifically to the use of human serum albumin, preferably produced by recombinant means for (1) treatment of psoriasis and eczema; (2) treatment of Acne or Acne related skin conditions, such as those induced by Epidermal Growth Factor Receptor (EGFR) inhibitors during cancer chemotherapy; (3) use in antiseptic topical aqueous solutions for the purpose of sanitizing the skin surface for better personal hygiene, surgical preparatory procedures and wound treatment; (4) use in protecting and fortifying skin against extreme environmental conditions, such as those produced by cold or arid environments, wind, radiation such as sun exposure, pollutants and premature aging of the skin; and (5) use as an additive in conventional topical pharmaceutical compounding ointments and gel bases to enhance the solubility absorption and the delivery of active pharmaceuticals.

The albumin-based gel of the invention can be formulated in various alcohols with additional bactericidal agents which will allow the albumin to be absorbed in the surface of the skin to enhance the natural barrier defense of the skin, while providing maximum penetration for the active anti-microbial, anti-psoriasis and/or ant-eczema ingredients by dramatically lowering the surface tension of the aqueous solution. Additionally, albumin's inherent small molecule binding properties can bind harmful bacterial fatty acids known to be a factor in certain types of acne related skin conditions. The albumin can be formulated with or without UV blockers in lotions, creams, water and/or alcohol based gels, oils and waxes, which will allow the albumin to be absorbed in the surface of the skin to effectively enhance the natural defense of the skin against extreme environmental factors, improve the skin barrier and hydration, along with many other inherent natural protective benefits of albumin. The albumin can be formulated in lotions and creams, preferably in water and alcohol based lotions and gels which control the pH and will allow the albumin to be absorbed in the surface of the skin to promote the delivery of antibacterial agents and other small molecules, while at the same time, enhancing the natural protective defense of the skin against toxic metabolites and pollutants, as a natural component of the skin, for the general relief and maintenance of the affected skin areas.

### BACKGROUND OF THE INVENTION

The serum albumins belong to a multigene family of proteins that includes alpha-fetoprotein and human group-specific component, also known as vitamin-D binding protein. The members of this multigene family are typically comprised of relatively large multi-domain proteins, and the serum albumins are the major soluble proteins of the circulatory system and contribute to many vital physiological processes. Serum albumin generally comprises about 50% of the total blood component by dry weight, and as such is responsible for roughly 80% of the maintenance of colloid osmotic blood pressure and is chiefly responsible for controlling the physiological pH of blood.

The albumins and their related blood proteins also play an extremely important role in the transport, distribution and metabolism of many endogenous and exogenous ligands in the human body, including a variety of chemically diverse molecules including fatty acids, amino acids, steroids, calcium, metals such as copper and zinc, and a plethora of pharmaceutical agents. The albumin family of molecules are generally thought to facilitate transfer many of these ligands across organ-circulatory interfaces such as the liver, intestines, kidneys and the brain, and studies have suggested the existence of an albumin cell surface receptor. See, e.g., Schnitzer et al., P.N.A.S. 85:6773 (1988). The albumins are thus involved in a wide range of circulatory and metabolic functions.

Human serum albumin (HSA) is a protein of about 66,500 kD protein and is comprised of 585 amino acids including at least 17 disulphide bridges. As with many of the members of the albumin family, human serum albumin plays an extremely important role in human physiology and is located in virtually every human tissue and bodily secretion. In fact, human serum albumin is one of the major extracellular proteins of skin, with approximately 40% of extravascular albumin located in the skin. Further, as indicated above, HSA has an outstanding ability to bind and transport a wide spectrum of ligands throughout the circulatory system including the long-chain fatty acids which are otherwise insoluble in circulating plasma. This same inherent binding and transport function also serves to protect the body from chemical toxins, such as harmful metabolic products (e.g., bilirubin) and various exogenous chemicals, such as pharmaceuticals. The atomic structure and particular details regarding the binding affinities of albumin and the specific regions primarily responsible for those binding properties have been previously determined as set forth, e.g., in U.S. Ser. No. 08/448,196, filed May 25, 1993, now U.S. Pat. No. 5,780,594 and U.S. Ser. No. 08/984,176, filed Dec. 3, 1997, now U.S. Pat. No. 5,948,609, said applications and patents incorporated herein by reference.

In the field of eczema, psoriasis, acne and acne related skin disorders; in the field of sanitizing, pre- and post-surgical skin treatment; in the field of pharmaceutical compounding ointment bases; and in the field of extreme environmentally protective lotions, bases and gels, such as those containing sunscreen; there has been no prior use of human albumin, specifically recombinant human albumin, as an active component of these treatments and formulations. Prior art products have focused on moisturizers, soap and cleansing agents, anti-wrinkling and other similar products which are formulated for cosmetic and skin cleaning. Thus, there is a significant need to develop safe, effective and markedly improved skin treatments for the applications and indications above and yet which can be used safely and effectively with a reduced risk of allergic reactions.

### SUMMARY OF THE INVENTION

Accordingly, it is thus an object of the present invention to provide novel skin treating compositions which utilize human serum albumin, and preferably which comprise recombinant human serum albumin.

It is further an object of the present invention to utilize recombinant human serum albumin as a dermatological agent in the treatment of psoriasis, eczema and related human skin disorders. It is still further an object of the present invention to utilize recombinant human serum albumin as a dermatological agent in the treatment of acne and acne like skin disorders such as those induced by cancer chemotherapeutics including, but not limited to those which are EGFR inhibitors, and other related human skin disorders.

It is yet a further an object of the present invention to utilize recombinant human serum albumin as an additive in skin sanitization solutions, such as those used for personal hygiene, professional health care, pre and post operative surgical preparation and wound treatment applications.

It is still further an object of the present invention to provide a composition for use in skin applications which are specifically designed to protect the skin from extreme environmental conditions, including those which include temperature extremes, low humidity, wind, sun exposure and other forms of radiation, to effectively reduce the risk of damaging effects which can accelerate aging (anti-aging) and promote cancer.

It is still further an object of the present invention to provide a composition for use in eye applications which are specifically designed to lubricate and/or promote the healing of eye after surgical or accidental trauma. Additionally, the albumin solution may be used as a base for addition of antibiotic or other therapeutic compounds or biological agents, such as human lysozyme.

It is even further an object of the present invention to provide albumin compositions which can be used as pharmaceutical compounding bases, such as creams, oils, lotions, gels or gel-based ointments, or any other form that is suitable for administration to skin which dissolves, delivers, or protects pharmaceutically active ingredients.

These and other objects are achieved by virtue of the present invention which provides a hypoallergenic dermatological compositions for sanitizing and treating the skin for a variety of skin conditions including those of the eye, which comprises serum albumin in an amount effective to achieve sanitizing or wound protection along with a suitable pharmaceutically acceptable vehicle, carrier or excipient that is compatible as a sanitizing or wound protective agent. The hypoallergenic albumin compositions of the invention may be in any suitable form for treating skin, such as a cream, oil, lotion, gel, gel-based ointment, balm and the like. The serum albumin compositions are preferably prepared using recombinant serum albumin and are useful in that they allow the albumin to be absorbed in the surface of skin so as to treat a variety of skin conditions when utilized as a sanitizing or dermatological treatment agent. The compositions of the present invention will provide sanitizing or dermatological compositions that can be used safely and effectively and with reduced likelihood of allergic reaction. If considered medically necessary, these compositions may be combined with other necessary ingredients such as steroids.

These and other features of the present invention as set forth in, or will become obvious from the detailed description of the preferred embodiments provided herein below.

### BRIEF DESCRIPTION OF THE DRAWING FIGURE

FIG. 1A shows Psoriasis or seborrheic dermatitis prior to treatment with the albumin alcohol gel; 1B shows Week 4, after treatment applied liberally once daily. Patient notes skin after treatment is normal, unlike steroid treated skin which left the skin thinner and atypical in appearance. Generally the dermatitis would not return for several weeks if treatment discontinued.

### DETAILED DESCRIPTION OF THE PREFERRED EMBODIMENTS

In accordance with the present invention, a skin treating composition is provided which comprises serum albumin in an amount effective to treat a variety of skin related disorders, or enhanced skin protection properties beneficial for extreme environmental conditions. In addition, methods are providing for treating a variety of skin conditions and for protecting the skin under certain conditions which comprise topical application of the compositions of the present invention in an amount necessary to treat said condition or to protect the skin in the manner described herein.

By effective amount is generally meant the amount of serum albumin used in the composition which will achieve or enhance a beneficial cosmetic or dermatological effect for skin, such as sanitization, treatment of a particular condition, moisturizing, etc., or would protect skin as described below, as would be readily understood by one skilled in the art. Accordingly, the actual amount of albumin used in the skin treating or protecting compositions of the present invention will vary greatly depending on the type of albumin used, the desired effect, and the type of dermatological agent, pharmaceutical compounding base, vehicle, carrier, excipient, or other suitable sanitizing or treatment base used in the composition, as would be recognized by one skilled in the art.

For example, in preparing compositions using an alcohol or water gel base as the carrier, concentrations of albumin preferably range from 1 mg/ml to 90 mg/ml of the gel base. However, depending on the specific purpose of the compositions or solutions of the invention, e.g., psoriasis treatment, acne treatment, eczema treatment, environmental protection, pharmaceutical compounding, etc., the amount of albumin used may vary, and can be adjusted based on the desired strength of the composition or solution. Accordingly, compositions in accordance with the invention can be prepared using albumin in concentrations as low as about 0.01 mg/ml or as high as about 250 mg/ml, which will again depend on the desired application and the nature of the carrier or base into which the albumin will be incorporated. Moreover, the albumin solutions can be prepared at pH conditions which promote the optimal functionality and stability of albumin, preferably at pH values between 4.5 and 9.0, but more preferably at pH 6.5 to 7.5.

As would also be recognized by one skilled in the art, the creams, lotions and balms used in accordance with the invention for the purposes of topically delivering therapeutics, nutrients, etc. to the skin for treating psoriasis and other skin disorders or protecting skin, may also include one or more natural oils. Conventional natural oils and fatty acids include palm oil, flax seed oil, grape seed oil, olive oil, corn oil, cod liver oil, fish oil, safflower seed oil, lemon oil, conjugated linoleic acids, palmitic acids and the like. Similarly, certain waxes natural and man made, could be used to formulate the desired bases. Conventional waxes include but are not limited to Candelilla wax, Soy wax, Bees wax, petroleum based wax, petrolatum, lanolin and the Compositions in accordance with the invention may also include important vitamins like vitamin C, D, A, K and E, or any other vitamins or other supplements commonly used in skin care products.

Furthermore, the incorporation of gelling agents which can improve product presentation and function for a variety of applications may also be used in accordance with the invention. Conventional gelling agents include one or more of the following, but are not limited to, hydroxyethylcelluose, carbomer, a polyethylene homopolymer, a polyethylene/vinyl acetate copolymer, a polyethylene/acrylic acid copolymer, azelaic acid, aloe vera, lecithin, thermoreversible polysaccarides, and cetylhydroxyethyl cellulose. Even further, where appropriate, it may be desired to add one or more UV blocking agents to protect the skin from UVA and UVB sun damage. These agents include but are not limited to Aminobenzoic acid (PABA), Avobenzone, Cinoxate, Dioxybenzone, Ecamsule (terephthalylidene dicamphor sulfonic acid), Homosalate, Methyl anthranilate, Octocrylene, Octyl methoxycinnamate, Octyl salicylate, Oxybenzone, Padimate O, Phenylbenzimidazole sulfonic acid, Sulisobenzone, Titanium dioxide, Trolamine salicylate, Zinc oxide. Still further, where appropriate, it may be desired to incorporate an anti-microbial such as: Silver sulfaldazine, tetracycline, sulfacetamide sodium/sulfur, bacitracin, polymyxin B, penicillin, amoxicillin, retpamulin, mupirocin, mafenide, sulfamethoxazole, trimethoprim, neomycin, sulfonamide, melocyclin, benzalkoniumchloride, accutane, doxycycline, etc.

Human albumin can be used to treat or enhance treatment of various forms of skin conditions, including psoriasis, eczema, acne, EGRF inhibitor-induced skin toxicities, etc. Vehicles and carriers commonly used in treating these conditions include, lotions, creams, bases, balms, as well as soaps and shampoos.

### Albumin: Theory of Action in Skin Treatment and Sanitization

As indicated above, It has been discussed previously that 40% of the body's extravascular albumin is located in one organ, the skin. Skin cells will not grow optimally in vitro without the presence of albumin in the growth medium. Thus, albumin promotes skin regrowth of healthy skin cells and healing. This property is no doubt imparted by two other functions of albumin, inherent in the circulatory system, control of the pH and binding an transporting an immense variety of nutrients to facilitate bioavailability (important vitamins, like Vitamin E and fatty acids which are otherwise insoluble). This important binding function also serves to protect the body since albumin sequesters foreign molecules which may be harmful, chemically react with DNA, etc. and in the case of plasma albumin, off loads them to the liver. In the skin, these bound molecules are captured by albumin (such as bilirubin, the yellow pigment found in the skin during jaundice).

In accordance with the present invention, albumin has been observed to penetrate and absorb into the dermis when applied through a variety of topical base formulations. Once in the dermis it can hydrate the dermis and also possibly the hypodermis, by osmotic concentration gradient effects. Once there, it may also contribute to the proper pH balance of the skin since this is also one of albumins functions in the circulatory system. However, it is important to recognize that the pH of the skin, typically 4.5 to 5.5, is at or near the isoelectric point of albumin (which varies widely depending on the type and quantity of ligands bound), a pH where albumin is insoluble and thus serving to enhance the skin barrier. An interesting supporting note is that the allergic reaction to pet dander, is to the albumin (ie., dog or cat albumin) in the dander.

When the albumin solutions are also used in conjunction with bacterial agents, such as ethanol, it is believed that the albumin molecule, surface saturated with ethanol, penetrates the epidermis a releases its ethanol payload or other anti-microbial over time in the dermis. It is also understood that ethanol contributes to delivery of therapeutic agents into the dermis. The dermis contains the oil sacs and hair follicles (sebaceous glands) that may be infected with bacteria. Albumin, even in small concentrations dramatically lowers the surface tension of aqueous solutions, allowing solutions deeper penetration through pores, hair follicles and other cracks and fissures in the skin. As a very large macromolecule, albumins diffusion coefficient is quite low, so it will reside in localized application areas for days or weeks, whereas, small molecules will diffuse away in a matter of minutes or hours.

Recently in a study by the National Institutes of Health, it was suggested that the cause of immune responses to certain skin disorders could be related to atypical bacterial flora, promoted by non-optimum skin pH conditions, etc. See, e.g., Grice et al., Topographical and Temporal Diversity of the Human Skin Microbiom, Science 324(5931):1190-1192, May 29, 2009), said article incorporated herein by reference. It is believed that the absorption of albumin has manifold benefits to help restore “normal” skin growth to the region, by 1) eliminating harmful bacteria, e.g., ethanol or another anti-microbial (such as clindamycin, peroxide, etc.) with albumin, 2) restoration of proper pH, nutrient delivery, etc. to skin cells. Skin cells do not grow optimally outside the body except in the presence of albumin. This theory has been supported by observations of reductions in psoriasis conditions which last for periods up to 4 weeks, then re-occur. The inventor has associated this time frame with the slow disappearance of the supplemented albumin. Once treatment is resumed, normal skin growth is re-established.

Finally, one of albumin's inherent functions is to sequester harmful agents, it is therefore a natural protectant from damaging environmental effects such as sun damage, cold, heat, wind and exposure to cytotoxic drugs such as cancer drugs or related toxicities, or other harmful exposure to toxic endogenous and exogenous ligands. Many of these compounds may also contribute to undesirable inflammatory reactions.

Alcohol gel based carriers combined with recombinant human serum albumin have been used effectively in many cases to treat acne, cancer treatment (e.g., EGRF inhibitor associated skin abnormalities), and psoriasis and eczema types of skin conditions (Example 1).

In the case of another embodiment, referred to herein as albumin supplemental therapy, the additional albumin can be used to provide added moisture and barrier protection while at the same time enhancing the capability of the skin to react with harmful metabolic or environmental toxins, an important inherent biological function of albumin described above. Moreover, in a related embodiment, the albumin supplement and/or drug delivery vehicle according to the invention can be in the form of a topical for use as a sunscreen to aid in the prevention of sunburn and limit the potential damage of free radicals to cause skin damage and potentially cancer, for the delivery of important nutrients for the normal growth and maintenance of skin.

Other conditions treatable by the albumin supplement and/or drug delivery vehicle of the invention include insect bites, chapped lips, bedsores and Herpes.

In addition, the albumin gel or lotion of the invention can constitute a drug delivery vehicle in the form of a topical treatment for acne, psoriasis, eczema and a variety of immune related skin disorders. The albumin supplement and/or drug delivery vehicle can also be in the form of a topical to promote wound sanitization and healing. If suitable, the albumin supplement and/or drug delivery vehicle in the form of a topical for treatment of a variety of skin disorder/conditions can also contain an anti-microbial agents, such as clindamycin, erythromycin, tetracycline, benzoyl peroxide, Silver sulfaldazine, tetracycline, sulfacetamide sodium/sulfur, bacitracin, polymyxin B, penicillin, amoxicillin, retpamulin, mupirocin, mafenide, sulfamethoxazole, trimethoprim, neomycin, sulfonamide, melocyclin, benzalkoniumchloride, accutane, doxycycline, benzoyl peroxide, human lysozyme, hen egg white lysozyme, chalorapsis lysozyme, acetic acid, ammonia, ethanol, isopropyl alcohol, phenoxyethanol, triclocarban, or anti-psoriasis, anti acne or anti eczema agents, such as zinc pyrithione, selenium suphide (selenium disulphide), Polylysine episilon, alcohol and salicylic acid or salts thereof, etc.

In another embodiment of the invention, a pharmaceutical compounding base can be prepared by incorporating an effective amount of albumin in a base material commonly used in sanitizing or skin treating compositions, e.g., alcohol gels. In a preferred embodiment, a 10-75% alcohol solution, preferably about 65%, containing about 1-90 mg/ml of recombinant serum albumin, preferably about 20 mg/ml, can be prepared and employed as a sanitizing solution for personal hygiene, surgical preparatory procedures, such as, pre and post operative incision treatment. The inventor theorizes that the composition of the invention will be useful in providing additional anti-microbial protection due to the enhanced skin penetration of the albumin mixture.

In the preferred embodiment, the compositions of the present invention can be prepared by direct addition of the albumin to the pharmaceutical base, dermatological agent or carrier, such as a cream, lotion or alcohol-gel base, and the albumin may be added in any appropriate form, e.g., solid, freeze-dried, liquid etc. With regard to the form of albumin useful in the compositions of the present invention, it is particularly preferred that human serum albumin be employed in these compositions, and preferably a recombinant serum albumin is used, such as those previously disclosed in U.S. Pat. No. 5,780,594 and U.S. Pat. No. 5,948,609, both of which are incorporated herein by reference. The albumin used may be in whole form or may be in the form of relevant fragments, such as particular domains, subdomains, etc., including those that have been disclosed in the patents referred to above. In addition, a modified or truncated human albumin such as disclosed in U.S. Pat. No. 6,787,636, incorporated herein by reference, may also be utilized in the invention. As set forth therein, the serum albumin may be one that has at least a one-amino acid truncation at its n-terminal end, or any other mutation at the n-terminal end which is sufficient to cause steric hindrance at the n-terminal end so as to reduce or eliminate the albumin's affinity to trace metals. Still other forms of albumin may also be suitable for certain applications.

Treatment compositions which include serum albumin in accordance with the invention, or the preferable recombinant human serum albumin, can be effectively formulated with a wide variety of conventional ingredients common to lotions, bases, ointments, creams, balms and the like which can comprise the vehicles, excipients or carriers in accordance with the invention, Example of these ingredients include, but are not limited to: Water, Ppg-15 Stearyl Ether, Oxidized Polyethylene, Stearyl Alcohol, Cetyl Betaine, Salicylic Acid, Distearyldimonium Chloride, Sodium Lauryl Sulfate, Cetyl Alcohol, Alcohol, Steareth-21, Cyclopentasiloxane, Niacinamide, Ethylene/Acrylic Acid. Copolymer, Dimethicone Crosspolymer, Propylene Glycol, Butylene Glycol, Panthenol, Peg-10 Dimethicone Crosspolymer, Tocopheryl Acetate Cyclopentasiloxane, Polymethylsilsesquioxane, Stearyl Dimethicone, Palmitoyl Pentapeptide-3, Cetearyl Alcohol, Polysorbate 60, Helianthus annuus (sunflower) seed oil, Butyrospermum parkii (shea butter), Neopentyl Glycol Diheptonate, Isododecane, Glycerine, Ricinus communis (castor) seed oil, Hydrogenated castor oil, Beeswax, Copernica cerifera (carnauba) wax, Prunus amygdalus dulcis (sweet almond) oil, Caprylic/Capric triglycerides, Lanolin, Cannabis sativa seed oil, Glycine, Conjugated linoleic acid (CLA), Sodium Chloride, Potassium phosphate, Isohexadecane, Petrolatum, Dihydroxyacetone, isopropyl Isostearate, Nylon 12, Aluminum Starch, Octenylsuccinate, Dimethiconol, Hydroxyethyl Acrylate/Sodium Acryloyldimethyl Tauxate Copolymer, Behenyl Alcohol, Erythrulose, Squalane, Benzyl Alcohol, Glyceryl Stearate, PEG 100 Stearate, Dicaprylyl Ether, Sodium Lactate, PEG 40 Stearate, Cyclopentasiloxane, Cyclohexasiloxane, Aluminum Starch, Mineral Oil, Phenoxyethanol, Panthenol, Stearic Acid, Dimethicone, Carbomer, Ceteareth-20, Sodium Hydroxide, Sodium. Citrate, Methylparaben, Propylparaben, Citric Acid, Ethylparaben, Glyceryl Stearate SE, Octyldodecanol, Alcohol Denatured, Myristyl Alcohol, Creatine, Ubiquinone, Carbomer, 1 Methylhydantoin 2 Imide, C12 15 Alkyl Benzoate, Glyceryl Monostearate, Diazolidinyl, Trolamine, Edetate Disodium, Xanthan Gum, White Petrolatum USP, Ceteareth-20, Malic Acid, Sodium Lactate, Xanthan Gum. NF, C10 30 Cholesterol/Lanesterol, Hexyldecanol, Isopropyl Myristate, Glyceryl Caprate, Carthamus Tinctorius (Safflower) Seed Oil (Safflower), Styrene/Acrylates Copolymer, PEG 5 Soy Sterol, 1,2 Hexanediol, Caprylyl Glycol, Ethylhexylglycerin, Triethanolamine, Grape seed oil, corn nil, coconut oil, olive oil, sodium palmitate, polyethylene (glycols (PEG), Candelilla wax, Soy wax, bees wax, petroleum based wax, Lecithin Gel (PLC) PEG-100 Polydimethylsiloxane, emulsifying wax, cyclomethicone, lemon nil, avocado oil, glycerin, vitamin C, vitamin D, Polylysine epsilon, propylparaben, flax seed oil, cod liver oil, petrolatum, and lanolin.

A general moisturizing lotion that can be utilized in accordance with the invention is set forth as follows:

Classification and Ingredients Information:

| INCI Names: | CAS Nos.: | Range Ingredient: |
|---|---|---|
| Water | 7732-18-5 | >30% to 100% |
| Cetearyl Alcohol | 8005-44-5 | >3% to 10% |
| Polysorbate 60 | 9005-67-8 | >3% to 10% |
| Caprylic/capric triglycerides | 8001-31-8 | >1% to 3% |
| Helianthus annuus (sunflower) | 8001-21-6 | >1% to 3% |
| seed oil | | |
| Butyrospermum parkii (shea | 68920-03-6 | >1% to 3% |
| butter) | | |
| Neopentyl Glycol Diheptonate | 68855-18-5 | >1% to 3% |
| Isododecane | 13475-82-6 | >1% to 3% |
| Glycerine | 56-81-5 | >1% to 3% |
| Prunus Amygoalus Dulcis (Sweet | 8007-69-0 | >1% to 3% |
| Almond) Oil | | |
| Vitis vinifera (grape) seed oil | 8024-22-4 | >1% to 3% |
| Tritcum vilgare (wheat germ) | 68917-73-7 | >1% to 3% |
| Glycol Stearate | 111-60-4 | >1% to 3% |
| Theobroma cacoa (cocoa) seed | 8002-31-1 | >0.3% to 1% |
| butter | | |
| Tocopheryl acetate (vitamin E) | 58-95-7 | >0.3% to 1% |
| Phenoxyethanol | 122-99-6 | >0.3% to 1% |
| Acrylates/C10-30 Alkyl Acrylate | 1310-73-2 | >0.1% to 0.3% |
| Crosspolymer | | |
| Sodium Hydroxide | | >0.1% to 0.3% |

A general lip or body balm that can be utilized in accordance with the invention is set forth as follows:

Classification and Ingredients Information:

| INCI Names: | CAS Nos.: | Range Ingredient: |
|---|---|---|
| Ricinus communis (castor) seed | 8001-79-4 | |
| oil & | | |
| Hydrogenated castor oil & | 8001-79-4 | |
| Beeswax & | 8012-89-3 | |
| Copernica cerifera (carnauba) | 8015-86-9 | >30% to 100% |
| wax | | |
| Prunus amygdalus dulcis (sweet | 8007-68-0 | >3% to 10% |
| almond) oil | | |
| Caprylic/Capric triglycerides | 8001-31-8 | >1% to 3% |
| Lanolin | 8006-54-0 | >0.3% to 1% |
| Tocopherol acetate | 58-95-7 | >0.3% to 1% |
| Cannabis sativa seed oil | — | 0.1% or less |

The present invention also contemplates the use of serum albumin as a sub or UV-blocking composition. A general list of ingredients that can be used as blocking agents along with albumin in accordance with the invention is set forth as follows:

### Sunscreen Ultra Violet Radiation FDA Approved Blocking Agents

Classification and Ingredients Information:

| Names: | CAS Nos.: | Range Ingredient: |
|---|---|---|
| Aminobenzoic acid (PABA) | — | > to 15% |
| Avobenzone | | > to 3% |
| Cinoxate | | > to 3% |
| Dioxybenzone | | > to 3% |
| Ecamsule (terephthalylidene | | > to 10% |
| dicamphor sulfonic acid) | | |
| Homosalate | | > to 15% |
| Methyl anthranilate | | > to 5% |
| Octocrylene | | > to 10% |
| Octyl methoxycinnamate | | > to 7.5% |
| Octyl salicylate | | > to 5% |
| Oxybenzone | | > to 6% |
| Padimate O | | > to 8% |
| Phenylbenzimidazole sulfonic acid | | > to 4% |
| Sulisobenzone | | > to 10% |
| Titanuim dioxide | | > to 25% |
| Trolamine salicylate | | > to 12% |
| Zinc oxide | | > to 25% |

Other sets of ingredient that can be utilized as the vehicle, excipient or carrier, or ingredients thereof, in accordance with the present invention, are as follows:

Water, Glycerin, Isohexadecane, Petrolatum, Dihydroxyacetone, Isopropyl Isostearate, Nylon 12, Aluminum Starch Octenylsuccinate, Dimethicone, Dimethiconol, Stearyl Alcohol, Hydroxyethyl Acrylate/Sodium Acryloyldimethyl Taurate Copolymer, Cetearyl Alcohol, Behenyl Alcohol, Erythrulose, Squalane, Benzyl Alcohol, Tocopheryl Acetate, Glyceryl Stearate, PEG 100 Stearate, Xanthan Gum, Disodium EDTA, Ethylparaben, Methylparaben, Propylparaben, Capric/Caprylic Stearic Triglyceride, Polysorbate 60, Fragrance (Parfum), Red 33, Yellow 5
Water, Glycerin, Urea, Glyceryl Stearate, Stearyl Alcohol, Dicaprylyl Ether, Sodium Lactate, Dimethicone, PEG 40 Stearate, Cyclopentasiloxane, Cyclohexasiloxane, Aluminum Starch. Octenylsuccinate, Lactic Acid, Xanthan Gum, Phenoxyethanol, Methylparaben, Propylparaben
Water, Mineral Oil, Glycerin, Capric/Caprylic Stearic Triglyceride, Cetearyl Alcohol, Phenoxyethanol, Panthenol, Cetearyl Alcohol, Stearic Acid, Dimethicone, Carbomer, Ceteareth-20, Sodium Hydroxide, Sodium Citrate, Methylparaben, Propylparaben, Citric Acid, Ethylparaben
Water, Glycerin, Mineral Oil, Glyceryl Stearate SE, Octyldodecanol, Alcohol Denatured, Stearic Acid, Dicaprylyl Ether, Dimethicone, Myristyl Alcohol, Creatine, Ubiquinone, Phenoxyethanol, Fragrance (Parfum), Carbomer, Sodium Hydroxide, 1 Methylhydantoin 2 Imide, Methylparaben, Propylparaben

### Active Ingredients: Octinoxate (7.5%) (Sunscreen), Octisalate (4%) (Sunscreen), Oxybenzone (3%) (Sunscreen)

Inactive Ingredients: Water Purified, C12 15 Alkyl Benzoate, Cetearyl Alcohol, Ceteareth 20, Cetearyl Alcohol, Glyceryl Monostearate, Propylene Glycol, White Petrolatum, Diazolidinyl Urea, Trolamine, Edetate Disodium, Xanthan Gum, Acrylates/C10 30 Alkyl Acrylate Crosspolymer, Vitamin E, Iodopropynyl Butylcarbamate, Fragrance (Parfum), Carbomer
Water USP (Purified), Glycerin USP, White Petrolatum USP, Cetearyl Alcohol (and), Ceteareth-20, Malic Acid, Sodium Lactate, Xanthan Gum NF, Dimethicone, C10 30 Cholesterol/Lanesterol Esters, Sodium Hydroxide NF, Cetyl Alcohol NF, Diazolidinyl Urea, Methylparaben, Cetyl Lactate, C12 15 Alcohols Lactate, Propylparaben NF, Cyclomethicone, Sodium PCA, Fragrance (Parfum)

### Active Ingredients: Homosalate (6%, Sunscreen), Octisalate (5%, Sunscreen), Avobenzone (3%, Sunscreen), Oxybenzone (3%, Sunscreen), Octocrylene (2.4%, Sunscreen)

Inactive Ingredients: Water, Hexyldecanol, Propylene Glycol, Stearic Acid, Isopropyl Myristate, Glyceryl Caprate, Glyceryl Stearate, PEG 100 Stearate, Cetearyl Alcohol, Carthamus Tinctorius (Safflower) Seed Oil (Safflower), Styrene/Acrylates Copolymer, PEG 5 Soy Sterol, 1,2 Hexanediol, Caprylyl Glycol, Ethylhexylglycerin, Dimethicone, Phenoxyethanol, Triethanolamine, Acrylates C10 30 Alkyl Acrylate Crosspolymer, Tetrasodium EDTA, Xanthan Gum, Soluble Collagen, Kinetin, Panthenol, Ascorbic Acid (Vitamin C), Hydrolyzed Elastin
Water, Glycerin, Stearyl Alcohol, Glyceryl Stearate, PEG 100 Stearate, Carthamus Tinctorius (Safflower) Seed Oil (Safflower), Ethylhexyl Hydroxystearate, Cetearyl Alcohol, Acrylates C10 30 Alkyl Acrylate Crosspolymer, Dimethicone, Panthenol, Glycine Soja Sterol (Soybean), Sodium Hydroxide, Kinetin, Aloe (Aloe Barbadensis) Leaf Juice, Tocopheryl Acetate, Ascorbic Acid (Vitamin C), Zea Mays Oil (Corn), Retinyl Palmitate, Cholecalciferol (Vitamin D3), Diazolidinyl Urea, Citric Acid, Methylparaben, Propylparaben
Water, Ppg-15 Stearyl Ether, Glycerin, Oxidized. Polyethylene, Stearyl Alcohol, Cetyl Betaine, Salicylic Acid, Distearyldimonium Chloride, Sodium. Lauryl Sulfate, Cetyl Alcohol, Alcohol, Steareth-21, Sodium Chloride, Behenyl Alcohol, Ppg-30, Steareth-2, Sodium Glycolate, Panthenol, Tocopheryl Acetate, Carnosine, Disodium Edta, Ascorbic Acid++, Fragrance, Iron Oxides.
Water, Cyclopentasiloxane, Glycerin, Niacinamide, Ethylene/Acrylic Acid Copolymer, Dimethicone, Dimethicone Crosspolymer, Propylene Glycol, Butylene Glycol, Panthenol, Peg-10 Dimethicone Crosspolymer, Tocopheryl Acetate, Palmitoyl Pentapeptide-3+, Sucrose Polycottonseedate, Stearyl Dimethicone, Titanium Dioxide, Bis-Peg/Ppg-14/14 Dimethicone Benzyl Alcohol, Peg-10 Dimethicone, Cetyl Ricinoleate, Allantoin, Carnosine+, Disodium Edta, Camellia Sinensis Leaf Extract, Peg-100 Stearate, Alumina, Citric Acid, Peg/Ppg-18/18 Dimethicone, Ethylparaben, Propylparaben, Methylparaben, Fragrance, Yellow 5, Red 40.

Cyclopentasiloxane, Water, Glycerin, Polymethylsilsesquioxane, Dimethicone, Niacinamide, Dimethicone Crosspolymer, Stearyl Dimethicone, Butylene Glycol, Panthenol, Propylene Glycol, Palmitoyl Pentapeptide-3, Tocopheryl Acetate, Camellia Sinensis Leaf Extract, Cucumis Sativus (Cucumber) Fruit Extract, Allantoin, Petrolatum, Cetyl Ricinoleate, Peg-10 Dimethicone Crosspolymer, Sucrose Polycottonseedate, Bis-Peg/Ppg-14/14 Dimethicone, Benzyl Alcohol, Peg-10 Dimethicone, Peg-100 Stearate, Ethylparaben, Methylparaben, Propylparaben, Disodium Edta, Triethoxycaprylylsilane, Mica, Titanium Dioxide, Iron Oxides.

As indicated above, the skin and hair treating albumin compositions of the present invention may take on a variety of forms which may be suitable for use as a skin treatment or dermatological agent. Such embodiments would include oils, moisturizing cream, hand lotions, shaving creams, gels, gel-based ointments, balms or any other application where the goal is treatment, sanitization or protective conditioning of skin. These forms are all well known in the art, as is well known the many conventional methods of preparing these dermatological forms which could be utilized to prepare the dermatological treating or protecting compositions in accordance with the invention which contain an effective amount of serum albumin, preferably in recombinant form. Again, all of these skin treatment forms in accordance with the invention will be comprised of an effective amount of albumin in a suitable base, i.e., an amount effective to achieve a desired treatment, sanitization, or protective dermatological purpose, as would be appropriate for the desired sanitary conditioning, skin treatment or other dermatological application.

In addition, in accordance with the present invention, the albumin compositions of the present invention may also be utilized to promote sanitization of skin to for personal hygiene, pre and post operative preparations and procedures, and other forms of hospital associated skin applications including, the treatment of surgical incision, surface wounds, etc., but not limited to the treatment of bedsores, and thus may also be used in sterile form for treating such conditions.

The advantages of the present invention are exemplified in that the sanitization, treatment of acne, psoriasis, eczema etc. using these albumin compositions and formulations will be enhanced and superior to formulations which do not include albumin because the compositions of the invention will allow for superior treatment of skin using the largest single natural extracellular protein component, namely serum albumin. In addition, these compositions are highly desirable because serum albumin, particularly human serum albumin, may be produced by recombinant methods so as to be extremely safe in that it is non-blood derived and thus free of animal-derived pathogens. Moreover, the presence of serum albumin in aqueous solutions dramatically lowers the surface tension of the aqueous phase, allowing albumin and its associated molecules to penetrate the skin in crevices, pores where the majority of bacteria, fungi or other microbial agents are located, etc. to enhance the treatment or sanitization potential of the albumin containing composition. The preferred compositions of the present invention will also be hypoallergenic so as to reduce or eliminate the possibility of causing an allergic reaction upon application of the compositions.

The compositions of the present invention can thus be made simply and inexpensively using conventional ingredients and methods currently used in the conventional preparation of skin protection agents, skin treatments or other dermatological products such as gels, lotions, balms or pharmaceutical compounding bases and the like. As indicated above, in the desired process, the albumin may be added directly to the desired dermatological base, such as by dissolving the serum albumin in a treatment base when it is desired to prepare a composition in accordance with the invention. However, as would be well known to those skilled in this art, there are numerous conventional processes that may be used to prepare the desired dermatological or pharmaceutical compounding agent, and any suitable variety of these techniques may be employed to prepare the desired compositions in accordance with the invention.

Similarly, the present compositions will be useful in a variety of dermatological purposes and the use of these forms of the invention will be by topical application in the conventional manner for the use of these products. The products obtained using the albumin compositions of the present invention will be superior to conventional treatment, protection, sanitization or moisturizing products in that they will have an enhanced treatment effect due to the use of the albumin.

In yet another embodiment, an albumin solution from 0.01 to 100 mg/ml is provided comprising a suitable eye solution, such as phosphate buffered saline as a base in the form of a topical solution for treatment of dry eye, lasik or other eye surgery, dry socket syndrome, promotion of healing involved with surgical or accidental trauma. Additionally, the albumin solution may be used as a base for addition of antibiotic or other therapeutic compounds or biological agents.

Accordingly, the compositions of the present invention can be used to create a wide variety of safe and effective pharmaceutical and other dermatological products which have superior qualities when used to treat a variety of skin disorders, protective or sanitization functions. In addition, the compositions will be hypoallergenic and thus will reduce or eliminate the likelihood of causing an allergic reaction when used.

It is thus submitted that the foregoing embodiments are only illustrative of the claimed invention and not limiting of the invention in any way, and alternative embodiments that would be obvious to one skilled in the art not specifically set forth above also fall within the scope of the claims.

In addition, the following examples are presented as illustrative of the claimed invention, and are not deemed to be limiting of the scope of the invention, as defined by the claims appended hereto, in any manner.

### EXAMPLES

### Example 1

Compositions in accordance with the present invention were prepared by dissolving various concentrations of recombinant human serum albumin in an inexpensive conventional lotion or gel base. These compositions were prepared by the direct addition of from 1 mg/ml to 90 mg/ml (20 mg/ml used in the clinical studies below) of recombinant human albumin in freeze-dried dissolved with gentle mixing in the alcohol gel base.

An Alcohol-based Gel
Classification and Ingredients Information:

| INCI Names: | CAS Nos.: | Range Ingredient: |
|---|---|---|
| De-ionized and purified Water | 7732-18-5 | >30% to 95% |
| SD Alcohol | | >5% to 90% |
| Propylene Glycol | | >0% to 10% |
| Tocopheryl acetate (vitamin E) | | >0% to 3% |
| Phenoxyethanol | | >0% to 3% |
| Hydroxyethylcellulose NF | | >0% to 3% |
| Sodium Hydroxide | | >0% to 1% |

Experimental use of this albumin alcohol gel was made in three separate human clinical studies focused on acne, EGFR inhibitor induced cutaneous toxicities.
Clinical Study Cutaneous Toxicities Associated with Epidermal Growth Factor Inhibitors
Phase II pilot study to assess the potential for topical human albumin to modulate the cutaneous toxicities associated with epidermal growth factor inhibitors in clinical practice.

### A. Study Design

The study is designed as a pilot investigation entering up to 15 patients who have experienced cutaneous toxicity with epidermal growth factor inhibitors. These patients will then be consented, offered human albumin topically to apply one area of involvement for approximately two to four weeks and then extended to other areas if they have experienced significant relief of symptoms or improvement of the rash.

The patients will be assessed for efficacy and toxicity every two weeks during the first six weeks, photographs of each area taken and compared with the untreated area so each patient will serve as their own control.

At the completion of the study, each patient will be offered up to another four weeks of study medication for topical self administration.

### B. Results

A total of 15 patients were enrolled in the study. Of these patients, one was removed from the study after the first week, based on the perception that the skin condition was worsening (it should be noted that this patient was on a dosing level of EGRF inhibitor at more than twice the other patients). Approximately 40% patients stated that the application of the gel was improving their condition, of these, three patients (or 21%) made extremely favorable statements of marked improvement (one of these patients had been treated several years earlier and remarked on a definite improvement over his previous experience with EGRF related acne). The remaining patients stated they observed no improvement or were noncompliant.

### Example 2

### Clinical Study: Psoriasis or Dermatitis

Exploratory studies on the treatment of psoriasis were conducted with one patient over the course of two years. The most effective treatment was achieved with albumin formulated in an alcohol base as given in EXAMPLE 1.

### Results:

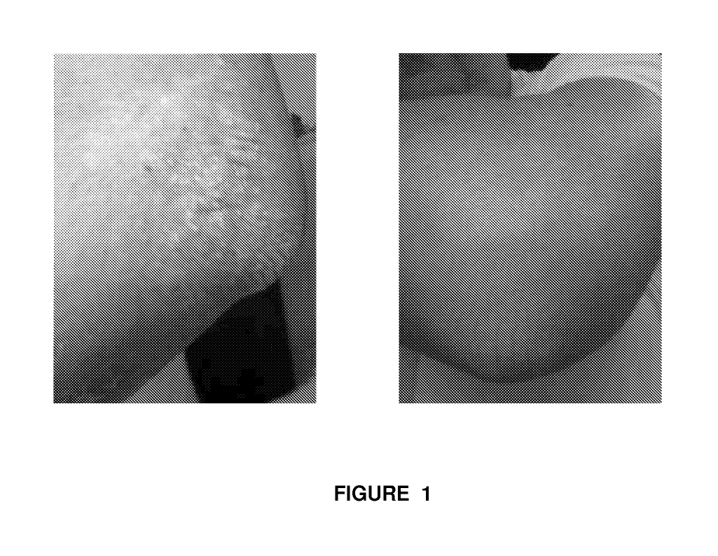

The patient's psoriasis affected areas were treated once daily with an alcohol base containing 20 mg/ml of recombinant human serum albumin. After day 3, the skin areas began crusting and scaling off, giving the appearance that the condition was worsening. This scaling was followed by the appearance of new skin which over the course of 3 weeks reverted to normal appearance (see FIG. 1). The condition would either 1) begin to re-appear after 2 to 4 weeks, if left untreated, or not return for several months.
The results were repeated with the same results on as series of experiments over the course of two years.

### Example 3

### Clinical Study: Acne

A total of approximately 50 patients exhibiting various forms of acne or acne-like skin conditions were supplied with 40 ml tubes of the albumin alcohol gel as described in EXAMPLE 1 and followed up with after a blank week period. Based on anecdotal evidence from patient surveys approximately 50% of the patients expressed improvement and interest in obtaining a continuing supply. Approximately 10% expressed marked improvement. Approximately 50% stated they observed no or little improvement.

### Example 4

Recombinant human serum albumin was added to the lotion base described below at 10 mg/ml. Experimental use of the compositions of the present invention was made with numerous individuals who used this lotion as applied to the hands for the treatment of very dry skin. All of the participants noted an immediate and distinctive difference in the texture and softness of the skin after drying their hands. All individuals noted a unique moisturizing and conditioning effect on the skin, without feeling the presence of an oil or grease residue. The following lotion base was used successfully as an example.

General moisturizing lotion:
Classification and Ingredients Information:

| INCI Names: | CAS Nos.: | Range Ingredient: |
|---|---|---|
| Water | 7732-18-5 | >30% to 100% |
| Cetearyl Alcohol | 8005-44-5 | >3% to 10% |
| Polysorbate 60 | 9005-67-8 | >3% to 10% |
| Caprylic/capric triglycerides | 8001-31-8 | >1% to 3% |
| Helianthus annuus (sunflower) | 8001-21-6 | >1% to 3% |
| seed oil | | |
| Butyrospermum parkii (shea | 68920-03-6 | >1% to 3% |
| butter) | | |
| Neopentyl Glycol Diheptonate | 68855-18-5 | >1% to 3% |
| Isododecane | 13475-82-6 | >1% to 3% |
| Glycerine | 56-81-5 | >1% to 3% |
| Prunus Amygoalus Dulcis (Sweet | 8007-69-0 | >1% to 3% |
| Almond) Oil | | |
| Vitis vinifera (grape) seed oil | 8024-22-4 | >1% to 3% |
| Tritcum vilgare (wheat germ) | 68917-73-7 | >1% to 3% |
| Glycol Stearate | 111-60-4 | >1% to 3% |
| Theobroma cacoa (cocoa) seed | 8002-31-1 | >0.3% to 1% |
| butter | | |
| Tocopheryl acetate (vitamin E) | 58-95-7 | >0.3% to 1% |
| Phenoxyethanol | 122-99-6 | >0.3% to 1% |
| Acrylates/C10-30 Alkyl Acrylate | 1310-73-2 | >0.1% to 0.3% |
| Crosspolymer | | |
| Sodium Hydroxide | | >0.1% to 0.3% |

### Example 5

Recombinant human serum albumin was added to the lotion base described below at 5 mg/ml. Experimental use of the compositions of the present invention was made with numerous individuals who used this lotion as applied to the hands for the treatment of very dry skin. All of the participants noted an immediate and distinctive difference in the texture and softness of the skin after drying their hands. All individuals noted a unique moisturizing and conditioning effect on the skin, without feeling the presence of an oil or grease residue. The following lotion base was used successfully as an example.

Classification and Ingredients Information:

| INCI Names: | CAS Nos.: | Range Ingredient: |
|---|---|---|
| Water | 7732-18-5 | >30% to 100% |
| Cetearyl Alcohol | 8005-44-5 | >3% to 10% |
| Polysorbate 60 | 9005-67-8 | >3% to 10% |
| Caprylic/capric triglycerides | 8001-31-8 | >1% to 3% |
| Helianthus annuus (sunflower) | 8001-21-6 | >1% to 3% |
| seed oil | | |
| Butyrospermum parkii (shea | 68920-03-6 | >1% to 3% |
| butter) | | |
| Neopentyl Glycol Diheptonate | 68855-18-5 | >1% to 3% |
| Isododecane | 13475-82-6 | >1% to 3% |
| Glycerine | 56-81-5 | >1% to 3% |
| Prunus Amygoalus Dulcis (Sweet | 8007-69-0 | >1% to 3% |
| Almond) Oil | | |
| Vitis vinifera (grape) seed oil | 8024-22-4 | >1% to 3% |
| Tritcum vilgare (wheat germ) | 68917-73-7 | >1% to 3% |
| Glycol Stearate | 111-60-4 | >1% to 3% |
| Theobroma cacoa (cocoa) seed | 8002-31-1 | >0.3% to 1% |
| butter | | |
| Tocopheryl acetate (vitamin E) | 58-95-7 | >0.3% to 1% |
| Phenoxyethanol | 122-99-6 | >0.3% to 1% |
| Acrylates/C10-30 Alkyl Acrylate | 1310-73-2 | >0.1% to 0.3% |
| Crosspolymer | | |
| Sodium Hydroxide | | >0.1% to 0.3% |

### Example 6

Recombinant human serum albumin was added to the lip balm base described below at 1 mg/ml. Experimental use of the compositions of the present invention was made with numerous individuals who used the lip balm for the treatment of chapped lips on individuals who frequently use lip balm. All of the participants noted a long lasting protective effect which was perceived as an important improvement over conventional lip balm.

A lip or body balm
Classification and Ingredients Information:

| INCI Names: | CAS Nos.: | Range Ingredient: |
|---|---|---|
| Ricinus communis (castor) seed | 8001-79-4 | |
| oil & | | |
| Hydrogenated castor oil & | 8001-79-4 | |
| Beeswax & | 8012-89-3 | |
| Copernica cerifera (carnauba) | 8015-86-9 | >30% to 100% |
| wax | | |
| Prunus amygdalus dulcis (sweet | 8007-68-0 | >3% to 10% |
| almond) oil | | |
| Caprylic/Capric triglycerides | 8001-31-8 | >1% to 3% |
| Lanolin | 8006-54-0 | >0.3% to 1% |
| Tocopherol acetate | 58-95-7 | >0.3% to 1% |
| Cannabis sativa seed oil | — | 0.1% or less |

What is claimed is:

1. A skin treatment composition comprising human serum albumin in an amount effective to treat a skin or scalp condition selected from the group consisting of psoriasis, eczema, acne, EGRF inhibitor related cutaneous skin toxicities and a suitable aqueous or alcohol-based gel, or suitable topical ointment and a pharmaceutically acceptable vehicle, carrier or excipient.

2. The composition according to claim 1 wherein the serum albumin comprises a recombinant serum albumin.

3. The composition according to claim 1 wherein the serum albumin comprises a serum albumin that has at least a one-amino acid truncation at its n-terminal end.

4. The composition of claim 1 wherein the vehicle, excipient or carrier is selected from the group consisting of creams, balms, lotions, gels, and ointments effective for the treatment of the targeted skin areas.

5. The composition of claim 4 wherein said vehicle, excipient or carrier includes an ingredient selected from the group consisting of: Water, Ppg-15 Stearyl Ether, Oxidized Polyethylene, Stearyl Alcohol, Cetyl Betaine, Salicylic Acid, Distearyldimonium Chloride, Sodium Lauryl Sulfate, Cetyl Alcohol, Alcohol, Steareth-21, Cyclopentasiloxane, Niacinamide, Ethylene/Acrylic Acid Copolymer, Dimethicone Crosspolymer, Propylene Glycol, Butylene Glycol, Panthenol, Peg-10 Dimethicone Crosspolymer, Tocopheryl Acetate Cyclopentasiloxane, Polymethylsilsesquioxane, Stearyl Dimethicone, Palmitoyl Pentapeptide-3, Cetearyl Alcohol, Polysorbate 60, Helianthus annuus (sunflower) seed oil, Butyrospermum parkii (shea butter), Neopentyl Glycol Diheptonate, Isododecane, Glycerine, Ricinus communis (castor) seed oil, Hydrogenated castor oil, Beeswax, Copernica cerifera (carnauba) wax, Prunus amygdalus dulcis (sweet almond) oil, Caprylic/Capric triglycerides, Lanolin, Cannabis sativa seed oil, Glycine, Conjugated linoleic acid (CLA), Sodium Chloride, Potassium phosphate, Isohexadecane, Petrolatum, Dihydroxyacetone, Isopropyl Isostearate, Nylon 12, Aluminum Starch Octenylsuccinate, Dimethiconol, Hydroxyethyl Acrylate/Sodium A′ Taurate Copolymer, Behenyl Alcohol, Erythrulose, Squalane, Benzyl Alcohol, Glyceryl Stearate, PEG 100 Stearate, Dicaprylyl Ether, Sodium Lactate, PEG 40 Stearate, Cyclopentasiloxane, Cyclohexasiloxane, Aluminum Starch, Mineral Oil, Phenoxyethanol, Panthenol, Stearic Acid, Dimethicone, Carbomer, Ceteareth-20, Sodium Hydroxide, Sodium Citrate, Methylparaben, Propylparaben, Citric Acid, Ethylparaben Glyceryl Stearate SE, Octyldodecanol, Alcohol Denatured, Myristyl Alcohol, Creatine, Ubiquinone, Carbomer, 1 Methylhydantoin 2 Imide, C12 15 Alkyl Benzoate, Glyceryl Monostearate, Diazolidinyl, Trolamine, Edetate Disodium, Xanthan Gum, White Petrolatum USP, Ceteareth-20, Malic Acid, Sodium Lactate, Xanthan Gum NF, C10 30 Cholesterol/Lanesterol, EDTA, Hexyldecanol, Isopropyl Myristate, Glyceryl Caprate, Carthamus Tinctorius (Safflower) Seed Oil (Safflower), Styrene/Acrylates Copolymer, PEG 5 Soy Sterol, 1,2 Hexanediol, Caprylyl Glycol, Ethylhexylglycerin, Triethanolamine, Grape seed oil, corn oil, coconut oil, olive oil, sodium palmitate, polyethylene glycols (PEG), Candelilla wax, Soy wax, Lecithin Gel(PLO), PEG-100 Polydimethylsiloxane, emulsifying wax, cyclomethicone, lemon oil, avocado oil, glycerin, vitamin C, vitamin D, Polylysine episilon, propylparaben, and flax seed oil

6. The skin treatment composition according to claim 1 comprising human serum albumin and one or more gelling agents.

7. The composition of claim 6 wherein the gelling agent is selected from the group consisting of hydroxyethylcelluose, carbomer, a polyethylene homopolymer, a polyethylene/vinyl acetate copolymer, a polyethylene/acrylic acid copolymer, azelaic acid, aloe vera, lecithin, thermoreversible polysaccarides, and cetylhydroxyethyl cellulose.

8. The composition according to claim 1 wherein the serum albumin comprises 1 to 100 mg/ml of a gel or solution comprised principally of water and/or alcohol.

9. The composition according to claim 8 wherein the alcohol or water-based gel or solution is from 1 to 90%.

10. The composition according to claim 9 wherein the serum albumin in the alcohol water-based gel or solution is at a concentration in the range of about 1 to 250 mg/ml.

11. The composition according to claim 2 wherein recombinant serum albumin is added to solution containing at least water, Cetearyl Alcohol and Polysorbate 60.

12. A method of treating a skin condition selected from the group consisting of psoriasis, eczema and acne comprising topically applying to the skin or scalp of a patient in need thereof the composition of claim 1 in an amount effective to treat said condition.

13. The skin treatment composition according to claim 1 comprising 1-90 mg/ml human serum albumin in a 1-90% alcohol solution.

14. A hypoallergenic skin treating composition for psoriasis eczema, or acne treatment comprising 1 to 90 mg/ml recombinant human serum albumin in a 1 to 90% alcohol gel or solution.

15. A hypoallergenic skin or scalp treating composition comprising 1 to 90 mg/ml of a modified human serum albumin that has at least one mutation at its n-terminal end sufficient to cause steric hindrance at the binding region VI and thereby reduce or eliminate the albumin's affinity to trace metals, and a suitable vehicle, excipient or carrier.

16. The hypoallergenic skin or scalp treating composition according to claim 15 wherein the vehicle, excipient or carrier comprises a 1-90% alcohol solution.

17. The skin treatment composition according to claim 1 wherein the pH is in a range from 4.5 to 9.0.

18. The skin treatment composition according to claim 1 further comprising an anti-microbial agent.

19. The composition of claim 18 wherein the anti-microbial is selected from the group consisting of clindamycin, erythromycin, tetracycline, benzoyl peroxide, Silver sulfaldazine, tetracycline, sulfacetamide sodium/sulfur, bacitracin, polymyxin B, penicillin, amoxicillin, retpamulin, mupirocin, mafenide, sulfamethoxazole, trimethoprim, neomycin, sulfonamide, melocyclin, benzalkoniumchloride, accutane, doxycycline, clindamycin, tetracycline, erythromycin, benzoyl peroxide, human lysozyme, hen egg white lysozyme, chalorapsis lysozyme, acetic acid, ammonia, ethanol, isopropyl alcohol, phenoxyethanol, and triclocarban.

20. The skin treatment composition according to claim 1 comprising human serum albumin and anti-psoriasis, anti-acne or anti-eczema agent.

21. The skin treatment composition according to claim 20 wherein the agent comprises a drug selected from the group of: zinc pyrithione, selenium suphide (selenium disulphide), and salicylic acid or salts thereof for the treatment of psoriasis, eczema or acne.

22. A skin treatment composition comprising human serum albumin in an amount effective to protect the skin from overexposure to the elements, environmental or metabolic, pharmaceutical or other pollutants, premature aging, and a suitable aqueous or alcohol-based gel or suitable topical ointment, lotion, cream, balm or gel and a pharmaceutically acceptable vehicle, carrier or excipient.

23. The composition according to claim 22 wherein the serum albumin comprises a recombinant serum albumin.

24. A method of protecting the skin from overexposure to the elements, environmental or metabolic, pharmaceutical or other pollutants, premature aging comprising topically administering to a human or animal patient in need thereof the composition of claim 22 in an amount effective to achieve said protection.

25. The skin treatment composition according to claim 22 comprising human serum albumin and gelling agent.

26. The composition of claim 25 wherein the gelling is selected from the group consisting of: hydroxyethylcelluose, carbomer, a polyethylene homopolymer, a polyethylene/vinyl acetate copolymer, a polyethylene/acrylic acid copolymer, azelaic acid, aloe vera, lecithin, thermoreversible polysaccarides, cetylhydroxyethyl cellulose.

27. The skin treatment composition according to claim 22 wherein the pH is in a range from 4.5 to 9.0.

28. The skin treatment composition according to claim 22 further comprising a sun or UV blocking agent.

29. A sun or UV blocking composition comprising human serum albumin and a sun or UV blocking agent.

30. The composition of claim 29 wherein the sun or UV blocking agent is selected from the group consisting of Aminobenzoic acid (PABA), Avobenzone, Cinoxate, Dioxybenzone, Ecamsule (terephthalylidene dicamphor sulfonic acid), Homosalate, Methyl anthranilate, Octocrylene, Octyl methoxycinnamate, Octyl salicylate, Oxybenzone, Padimate O, Phenylbenzimidazole sulfonic acid, Sulisobenzone, Titanium dioxide, Trolamine salicylate and Zinc oxide.

32. The composition of claim 29 further comprising a physiologically acceptable vehicle, excipient or carrier.

33. The composition of claim 32 wherein the vehicle, excipient or carrier is selected from the group consisting of: creams, balms, lotions, gels, and ointments effective for the protection of the targeted skin areas.

34. The composition of claim 32 wherein the vehicle, excipient or carrier contains an ingredient is selected from the group consisting of Water, Ppg-15 Stearyl Ether, Oxidized Polyethylene, Stearyl Alcohol, Cetyl Betaine, Salicylic Acid, Distearyldimonium Chloride, Sodium Lauryl Sulfate, Cetyl Alcohol, Alcohol, Steareth-21, Cyclopentasiloxane, Niacinamide, Ethylene/Acrylic Acid Copolymer, Dimethicone Crosspolymer, Propylene Glycol, Butylene Glycol, Panthenol, Peg-10 Dimethicone Crosspolymer, Tocopheryl Acetate Cyclopentasiloxane, Polymethylsilsesquioxane, Stearyl Dimethicone, Palmitoyl Pentapeptide-3, Cetearyl Alcohol, Polysorbate 60, Helianthus annuus (sunflower) seed oil, Butyrospermum parkii (shea butter), Neopentyl Glycol Diheptonate, Isododecane, Glycerine, Ricinus communis (castor) seed oil, Hydrogenated castor oil, Beeswax, Copernica cerifera (carnauba) wax, Prunus amygdalus dulcis (sweet almond) oil, Caprylic/Capric triglycerides, Lanolin, Cannabis sativa seed oil, Glycine, Conjugated linoleic acid (CLA), Sodium Chloride, Potassium phosphate, Isohexadecane, Petrolatum, Dihydroxyacetone, Isopropyl Isostearate, Nylon. 12, Aluminum Starch, Octenylsuccinate, Dimethiconol, Hydroxyethyl Acrylate/Sodium Acryloyldimethyl Taurate Copolymer, Behenyl Alcohol, Erythrulose, Squalane, Benzyl Alcohol, Glyceryl Stearate, PEG 100 Stearate, Dicaprylyl Ether, Sodium Lactate, PEG 40 Stearate, Cyclopentasiloxane, Cyclohexasiloxane, Aluminum Starch, Mineral Oil, Phenoxyethanol, Panthenol, Stearic Acid, Dimethicone, Carbomer, Ceteareth-20, Sodium Hydroxide, Sodium Citrate, Methylparaben, Propylparaben, Citric Acid, Ethylparaben, Glyceryl Stearate SE, Octyldodecanol, Alcohol Denatured, Myristyl Alcohol, Creatine, Ubiquinone, Carbomer, 1 Methylhydantoin 2 Imide, C12 15 Alkyl Benzoate, Glyceryl Monostearate, Diazolidinyl, Trolamine, Edetate Disodium, Xanthan Gum, White Petrolatum USP, Ceteareth-20, Malic Acid, Sodium Lactate, Xanthan Gum NE, C10 30 Cholesterol/Lanesterol, Hexyldecanol, Isopropyl Myristate, Glyceryl Caprate, Carthamus Tinctorius (Safflower) Seed Oil (Safflower), Styrene/Acrylates Copolymer, PEG 5 Soy Sterol, 1,2 Hexanediol, Caprylyl Glycol, Ethylhexylglycerin, Triethanolamine, Grape seed oil, corn oil, coconut oil, olive oil, sodium palmitate, polyethylene glycols (PEG), Candelilla wax, Soy wax, bees wax, petroleum based wax, Lecithin Gel(PLO), PEG-100 Polydimethylsiloxane, emulsifying wax, cyclomethicone, lemon oil, avocado oil, glycerin, vitamin C, vitamin. D, Polylysine epsilon, propylparaben, flax seed oil, cod liver oil, petrolatum, and lanolin.

35. The composition according to claim 22 wherein recombinant serum albumin is added to solution containing at least water, Cetearyl Alcohol and Polysorbate 60.

36. The composition according to claim 22 wherein the serum albumin comprises a serum albumin that has at least a one-amino acid truncation at its n-terminal end.

37. A method of treating the skin comprising topically applying the composition of claim 22 to skin in an amount effective for protecting targeted areas of the skin against environmental factors.

38. The skin treatment composition according to claim 22 comprising 1-90 mg/ml human serum albumin in a 1-90% alcohol solution.

39. A hypoallergenic skin protecting composition comprising 1 to 90 mg/ml recombinant human serum albumin in a 1 to 90% alcohol gel or solution.

40. The composition according to claim 22 wherein the serum albumin in the alcohol gel or solution is at a concentration in the range of about 1 to 250 mg/ml.

41. A method of protecting the skin against extreme environmental factors comprising applying to said skin the composition of claim 22 in an amount effective to protect the targeted skin areas.

42. A skin treatment composition according to claim 29 comprising 1-90 mg/ml human serum albumin in a 1-90% alcohol solution.

43. A hypoallergenic skin protecting composition comprising 1 to 90 mg/ml of a modified human serum albumin that has at least one mutation at its n-terminal end sufficient to cause steric hindrance at the binding region VI and thereby reduce or eliminate the albumin's affinity to trace metals, in a 1-90% alcohol solution.

44. The skin protecting composition according to claim 43 comprising 1-60 mg/ml human serum albumin in a 5-70% alcohol gel or solution.

45. The skin protecting composition according to claim 43 further comprising a carbomer in an amount effective to form an alcohol or water-based gel solution.

46. The skin treatment composition according to claim 43 wherein the pH is in a range from 4.5 to 9.0.

47. A skin treatment composition comprising human serum albumin in an amount effective to enhance the sanitization of skin for a group consisting of personal hygienic, pre or post surgical, or wound treatment purposes and a suitable aqueous or alcohol-based gel or topical ointment and one or more suitable antimicrobial agents, vehicles, carriers or excipients.

48. A method of preparing the skin for surgical incision comprising topically administering serum albumin with an antimicrobial in a dermatological agent, vehicle, carrier or excipient in an amount affective to sanitize the targeted skin areas.

49. A method of treating skin wounds, bed sores or damaged skin comprising applying to said skin the composition of claim 43 in an amount effective to sanitize the wound surface.

50. The composition according to claim 43 wherein the albumin is recombinant human serum albumin.

51. The skin treatment composition according to claim 43 comprising human serum albumin and gelling agent.

52. The skin treatment composition according to claim 51 wherein the gelling is selected from the group consisting of: hydroxyethylcelluose, carbomer, a polyethylene homopolymer, a polyethylene/vinyl acetate copolymer, a polyethylene/acrylic acid copolymer, azelaic acid, aloe vera, lecithin, thermoreversible polysaccarides, cetylhydroxyethyl cellulose.

53. The skin treatment composition according to claim 43 comprising recombinant human serum albumin and an anti-microbial.

54. The composition of claim 53 wherein the anti-microbial is selected from the group consisting of: clindamycin, erythromycin, tetracycline, benzoyl peroxide, Silver sulfaldazine, tetracycline, sulfacetamide sodium/sulfur, bacitracin, polymyxin B, penicillin, amoxicillin, retpamulin, mupirocin, mafenide, sulfamethoxazole, trimethoprim, neomycin, sulfonamide, melocyclin, benzalkoniumchloride, accutane, doxycycline, benzoyl peroxide, human lysozyme, hen egg white lysozyme, chalorapsis lysozyme, acetic acid, ammonia, ethanol, isopropyl alcohol, phenoxyethanol, triclocarban.

55. An eye treating composition comprising human serum albumin in an amount effective to lubricate and/or promote the healing of eye after surgical or accidental trauma and a vehicle, excipient or carrier suitable for use in the eye.

56. An eye treatment composition according to claim 55 wherein the human serum albumin is recombinant.

57. An eye treatment composition according to claim 55 further comprising an anti-microbial agent.

58. A skin treatment composition comprising of human serum albumin in an amount effective to be used in pharmaceutical compounding bases, such as creams, oils, lotions, gels or gel-based ointments, balms or any other form that is suitable for administration to skin which promotes dissolving, delivering, and/or protecting pharmaceutically active ingredients.
